(19) United States
(12) Patent Application Publication
(10) Pub. No.: US 2022/0023195 A1
(43) Pub. Date: Jan. 27, 2022

(54) COMPOSITIONS AND METHODS INCLUDING SAPINDUS AND GLEDITSIA EXTRACTS FOR PERSONAL CARE

(71) Applicant: Samantha Beauty Corp., Garden Grove, CA (US)

(72) Inventors: Tycho Joseph Speaker, Santa Cruz, CA (US); Elena Sik, Gilbert, AZ (US); Samantha Bao Anh Nguyen, Westminster, CA (US)

(73) Assignee: Samantha Beauty Corp., Garden Grove, CA (US)

(21) Appl. No.: 17/312,304

(22) PCT Filed: Dec. 13, 2018
(86) PCT No.: PCT/US2018/065369

§ 371 Date: Jun. 9, 2021

(51) Int. Cl. A61K 8/9789; A61K 8/9794; A61K 8/92; A61Q 5/02; A61Q 5/06; A61Q 5/12; A61Q 5/00; A61Q 19/10

(52) U.S. Cl. CPC A61K 8/9789; A61K 8/9794; A61K 8/922; A61Q 5/02; A61K 2800/805; A61Q 5/12; A61Q 5/002; A61Q 19/10; A61K 2800/5922; A61Q 5/06

(57) ABSTRACT

Provided is a composition suitable for treating hair, skin or nails that includes chum ket extract, soapnut extract, and at least one oil or extract selected from the group consisting of gac (Momordica cochinchinensis) oil, tamanu nut oil, lemon peel oil, pomelo peel oil (Citrus maxima peel oil), jojoba oil, macadamia oil, coconut oil, sunflower seed oil, argan oil, turmeric root extract, and dandelion root extract. The composition is useful for personal care, especially for treating hair, skin or nails.

### CROSS-REFERENCE TO RELATED APPLICATIONS

This is the national phase of International Application No. PCT/US2018/065369, filed 13 Dec. 2018. The disclosure of this application is incorporated herein by reference in its entirety for all purposes.

### FIELD OF THE INVENTION

The present invention relates to compositions and methods for personal care, in particular for treating hair, skin and nails. More particularly, the present disclosure relates to compositions and methods for strengthening and smoothing damaged hair fibers, such as hair damaged by normal wear and weathering or hair styling treatments.

### BACKGROUND OF THE INVENTION

Hair damage is an essentially unavoidable problem that can result from any or all of a variety of mechanical, chemical and other processes encountered in the course of daily life. Hair that is subjected to styling treatments such as straightening, curling, coloring, or bleaching typically undergoes substantial damage inherent to those processes. Additionally, swimming or outdoor activities leading to wear and tangling that can result in damage that is compounded by mechanical grooming required to remove the tangle and disarray. Damage to the hair surface creates conditions such as predilection to tangling and flyaway that lead to more damage in a downward spiral, culminating in breakage.

Typical hair care compositions include materials to improve shine and to provide smoothness to hair, and can temporarily mask damage. Other compositions may include polymeric materials that non-selectively coat and thicken the hair, and such “build-up” may not be desirable or may negatively change the feel or texture of the hair. Unfortunately, if the underlying damage is not repaired, the cycle of progressive damage and weakening will continue, eventually leading to hair breakage. Some ingredients are available to hair care formulators that can produce repair without buildup, such as those taught in US Patent Application Publication 2017/0348204 “Products Containing Charged Polymer Complex Microcapsules and Method of Use”, but commercial availability of such materials is limited to selected oils and while these modern materials can be made from natural components, they lack a historical context of use, particularly across consumer populations of varying ethnicity.

Current compositions and methods claiming to strengthen hair further utilize synthetic materials that are undesirable to many consumers. Many consumers have concern about toxicity or environmental impacts of synthetic materials, especially polymers, and prefer ingredients derived from natural materials and/or those with a long history of human use.

There is thus a need for compositions and methods for strengthening hair and preventing breakage that are derived from natural materials traditionally used for hair care. In addition, there remains a need for compositions suitable for treating skin and nails.

### SUMMARY OF THE INVENTION

The present invention relates to a composition suitable for treating hair, skin or nails. The compositions described herein include two materials traditionally used in Vietnam for hair care, and each found in nature, but normally used separately. Extracts of chum khet (Gleditsia sp.) seeds are traditionally used in Southern Vietnam while extracts of soapnut (Sapindus sp.) seeds are traditionally used in Northern Vietnam. The compositions include both of these extracts, gaining the benefit of each component as well as the combined action of the paired materials. In addition, the composition of also includes at least one oil or extract from a specified group, many of which are commonly used in hair care compositions in Vietnam.

Thus, in one aspect, the present invention relates to a composition suitable for treating hair, skin or nails, including, i.e., comprising:
a chum ket extract;
a soapnut extract; and
at least one oil or extract selected from the group consisting of gac (Momordica cochinchinensis) oil, tamanu nut oil, lemon peel oil, pomelo peel oil (Citrus maxima peel oil), jojoba oil, macadamia oil, coconut oil, sunflower seed oil, argan oil, turmeric root extract, and dandelion root extract.

In another example, the composition includes at least two of the oils or extracts described above.

In another example, the composition includes at least three of the oils or extracts described above.

In another example, the composition includes at least four of the oils or extracts described above.

In another example, the composition further comprises at least one alginate/chitosan microcapsule.

In another example, the composition includes at least 0.1% by weight of the oil or extract.

In another example, the composition includes at least 0.5% by weight of the oil or extract.

In another example, the composition includes at least 1% by weight of the oil or extract.

In another example, the composition includes at least 0.01% by weight, in total, of the chum ket extract and the soapnut extract.

In another example, the composition is a shampoo.

In another example, the composition is a conditioner.

In another example, the composition is a leave-in hair product.

In another example, the composition is a styling product.

In another example, the composition includes at least one of a body oil, cream, lotion, or butter.

Another aspect of the present invention is a method of preparing the composition described above by combining the chum ket extract, soapnut extract, and the at least one oil or extract as described above.

Another aspect of the present invention is a method of treating hair comprising applying an effective amount of the composition described above to the hair.

Another aspect of the present invention is a method of strengthening hair comprising applying an effective amount of the composition described above to the hair.

Another aspect of the present invention is a method of reducing and/or preventing hair breakage comprising applying an effective amount of the composition as described above to the hair.

Another aspect of the present invention is a method of treating skin, comprising applying an effective amount of the composition described above to the skin.

Another aspect of the present invention is a method of treating nails, comprising applying an effective amount of the composition described above to the nails.

### DETAILED DESCRIPTION OF THE INVENTION

Representative embodiments of the invention are described herein, although the invention is not intended to be limited to these embodiments. All percentages of components of the composition described herein refer to weight percentages, based on the total weight of the composition, unless otherwise noted.

The chum ket and soapnut extract components of the invention provide, among other benefits, foaming and cleansing activity. Combinations of the two ingredients may in some embodiments of the present invention provide additional foaming power due to synergistic action of the components.

The chum ket and soapnut extract may be obtained from a variety of sources. A suitable Sapindus extract that can be used in the present invention is supplied by Sabinsa Corp., East Windsor, N.J., as SAPINDIN® (SOAP UT EXTRACT). Alternatively, raw soap nuts can be crushed and extracted into hot water. One kg of soap nuts can be extracted into 10 kg of 100° C. water to produce an extract that may be used herein.

A suitable Gleditsia extract that can be used in the present invention is supplied by Greentech Biotechnologies, St Beauzire, France, as GREENTECH™ GLEDITSCHIA. Alternatively, raw Gleditsia seedpods can be roasted at 150° C. for 10 minutes, then extracted into 100° C. water. One kg of Gleditsia can be extracted into 10 kg of 100° C. water to produce an extract that may be used herein.

The composition of the present invention may contain 0.01% to 25%, in total, of the chum set and soapnut extracts. This range includes all specific values and subranges therebetween, such as 0.02%, 0.05%, 0.1%, 0.2%, 0.5%, 1.0%, 2.0%, 5.0%, 10%, 12%, 15%, 18%, 20%, and 22% by weight.

The composition also includes at least one oil or extract selected from the group consisting of gac (Momordica cochinchinensis) oil, tamanu nut oil, lemon peel oil, pomelo peel oil (Citrus maxima peel oil), jojoba oil, macadamia oil, coconut oil, sunflower seed oil, argan oil, turmeric root extract, and dandelion root extract. Thus, the composition may contain one, two, three, four, five or more of these oils or extracts, in any combination.

The composition may contain from 0.1% to 20% by weight, in total, of the oils and extracts described above. This range includes all specific values and subranges therebetween, such as 0.2%, 0.5%, 1.0%, 2.0%, 5.0%, 10%, 12%, 15%, and 18% by weight. Preferred ranges are from 0.5% to 5% and 1% to 3% by weight.

The recited oil components of the present invention provide shine, sealing, and strengthening benefits in some embodiments of the present invention. Further, in some embodiments, the combination of these oils provides benefits that exceed those provided by the single oils alone. In a non-limiting example of the synergistic benefit of the oil combinations, shine and strength of damaged hair samples treated with a combination comprising pomelo, jojoba, and argan oil were improved compared to control samples treated with any one of these oils alone. It will be apparent to one of ordinary skill in hair care formulation that the properties of blends of oils can extend beyond the properties of any individual oil in the blend.

Without being bound to any particular interpretation, the pomelo oil, being of very low viscosity and considerable volatility, may provide particular benefit in reducing the viscosity of the oil system and aiding penetration into very small spaces within damaged regions of hair. Similarly, without being bound by any particular theory, the jojoba oil, being a wax-like monoester may contribute an enduring waterproofing property. Also, without being bound by any theory, the gac oil, being highly viscous and high in antioxidants, may contribute both substantivity and antioxidant protective benefits. In combination the gac, jojoba and pomelo oil components may better deliver the waterproofing properties of the jojoba oil and antioxidant benefits of the gac oil into the smallest regions of hair damage. Similarly other combinations of the recited oil components may provide benefits unique to the combinations that are not realized in the component oils taken singly.

In another aspect the present invention discloses use of the traditional oils and extracts described above in encapsulated form, providing both selective deposition onto damaged hair surfaces and also a synergistic action by which the selected oils and plant extracts act in conjunction with the deposited capsules to produce a semi-permanent repair of superficial damage.

In some embodiments, at least one of the oils or extracts described above is incorporated into an alginate/chitosan microcapsule (for example, Vegabead Argan, Earth Supplied Products, Naples, Fla.).

Another aspect of the present invention is a method of preparing the composition described above by combining the chum ket extract, soapnut extract, and the at least one oil or extract as described above. The components of the composition may be mixed with stirring, for example.

Another aspect of the present invention is a method of treating hair comprising applying an effective amount of the composition described above to the hair.

Another aspect of the present invention is a method of strengthening hair comprising applying an effective amount of the composition described above to the hair.

Another aspect of the present invention is a method of reducing and/or preventing hair breakage comprising applying an effective amount of the composition as described above to the hair.

In preferred examples of the invention, the hair treated with the composition is human hair. However, the composition may also be useful in veterinary applications. In such embodiments, the composition is applied to animal hair, e.g., dog, cat or horse hair. The amount of the composition used to treat hair as described above may vary widely. Preferably, the hair is coated with a layer of the composition.

Another aspect of the present invention is a method of treating skin, comprising applying an effective amount of the composition described above to the skin. In preferred examples of the invention, the skin treated with the composition is human skin although veterinary use is also within the scope of the invention. The amount of the composition used to treat skin as described above may vary widely. Preferably, the skin is coated with a layer of the composition.

Another aspect of the present invention is a method of treating nails, comprising applying an effective amount of the composition described above to the nails. In preferred examples of the invention, the nails treated with the composition are human nails, including fingernails and toenails. As with the embodiments described above, the composition may be used in veterinary applications to treat animals. The amount of the composition used to treat nails as described above may vary widely. Preferably, the nails are coated with a layer of the composition.

The composition of the present invention may contain water. The composition may include an oil and a water phase, and either of these phases may be dispersed or emulsified in the other, continuous phase. Either the oil or the water phase, or both phases, may further comprise other specific components, including without limitation the extracts discussed above. Specific forms of the composition are described below.

### Hair/Skin Oil

In one embodiment, the compositions includes at least one of a hair oil, body oil, cream, lotion, butter, or similar product format, further comprising some or all of the oils described herein. Optionally, such compositions may comprise both an oil and a water phase, and either of these phases may be dispersed or emulsified in the other, continuous phase. Either the oil or the water phase, or both phases may further comprise other specific components, including without limitation the extracts recited in the present invention. Such product forms may be applied to hair to provide a range of benefits of including without limitation moisturizing, detangling, strengthening, smoothing, increasing shine, body, curl, straightness, or to style or protect hair from damage. These product forms may also be applied to skin or nails to provide a range of benefits, including without limitation moisturizing, smoothing, softening, strengthening, soothing, or protecting from oxidative damage.

### Shampoo/Bodywash

In one embodiment, the present invention can comprise a shampoo, bodywash or similar cleansing base intended to clean hair as well as to deliver the benefits of strengthening and reducing and/or preventing breakage of damaged hair. Compositions of the recited oils and plant extracts of the present invention and optionally including at least one of the named oils further comprising alginate/chitosan microcapsules are readily formulated into shampoo products further comprising aqueous solutions of any of a wide variety of surfactant materials available to modern hair formulators. Without limitation, such surfactant materials include anionic, cationic, non-ionic and amphoteric surfactants as well as structured surfactant systems, as well as co-dispersed immiscible oils such as silicones or silicone derivatives, panthenol, humectants such as glycerin and glycols, or combinations thereof. In some embodiments the surfactants may be limited to exclude sulfates or to surfactants that are derived from plant sources. In some embodiments, the shampoo may be formulated to fall within a specific pH range, for example pH 4.5-5.5. In other embodiments, the shampoo may be formulated to be clear or opaque.

### Conditioner

In one embodiment, the includes a conditioner, or similar rinse-off base intended to provide conditioning benefits such as shine, detangling, lubricity, frizz-control, and/or other desired characteristics. Compositions of the oils and plant extracts described above and, optionally, including at least one of the oils described above and further comprising alginate/chitosan microcapsules are readily formulated into conditioning products further comprising aqueous solutions of any of a wide variety of conditioning agents available to modern hair formulators. Without limitation, such materials include anionic, cationic, non-ionic and amphoteric materials as well as surfactants, polymers, co-dispersed immiscible oils such as silicones or silicone derivatives, panthenol, humectants such as glycerin and glycols, or combinations thereof. In some embodiments the conditioning agents may be limited to exclude sulfates or to materials that are derived from plant sources. In some embodiments the product may be formulated to fall within a specific pH range, for example pH 4.5-5.5. In other embodiments the product may be formulated to be clear or opaque.

### Leave-In Products

In another embodiment, the composition is a product intended to be applied and left upon the hair for an extended time until the next shampoo. Such leave-in products can provide styling benefits such as hold, curl, shine, detangling, lubricity, frizz-control, and/or other desired characteristics. Compositions of the recited oils and plant extracts of the present invention and optionally including at least one of the named oils further comprising alginate/chitosan microcapsules are readily formulated into leave-in products further comprising aqueous solutions of any of a wide variety of styling and benefit agents available to modern hair formulators. Without limitation, such materials include anionic, cationic, non-ionic and amphoteric materials as well as polymers, surfactants, co-dispersed immiscible oils such as silicones or silicone derivatives, panthenol, humectants such as glycerin and glycols, or combinations thereof. In some embodiments the materials may be limited to exclude sulfates or to materials that are derived from plant sources. In some embodiments, the product may be formulated to fall within a specific pH range, for example pH 4.5-5.5. In other embodiments, the product may be formulated to be clear or opaque.

### EXAMPLES

The following non-limiting illustrative examples demonstrate generalized embodiments of the present invention such that one of ordinary skill in the art of hair care formulation could reproduce the product qualities described herein using commonly practiced methods of manufacture.

### Example 1: Model Shampoo/Bodywash Formulation

Phase A: sodium lauroyl methyl taurate 8%, sodium lauroyl sarcosinate 10%, cocamidopropyl betaine 10%, Phase B: behentrimonium chloride 1%, Phase C: lauramidopropylamine oxide 8%, Phase D: vegabead argan 1% (ESP, Naples, Fla.), preservative and fragrance as desired, Phase E: gac oil 0.1%, tamanu oil 0.1%, pomelo peel oil 0.1%, jojoba oil 0.5%, macadamia oil 0.1%, coconut oil 1.0%, sunflower seed oil 0.1%, turmeric root extract 0.1%, dandelion root extract 0.1%, chum ket extract 0.1%, soapnut extract 0.1%, Phase F: viscarin PC-389 carrageenan 0.1%, xanthan gum 0.1%, deionized water q.s. 100%. Instructions: Phase F is combined with shear and heated to 75° C. to hydrate the gums fully, then heating is discontinued. Phase B is dispersed in Phase F while still hot. Phases A and C are added to the cooling batch. Once the batch has cooled below 45° C. phases D and E are added and dispersed with moderate shear.

### Example 2: Model Conditioner Formulation

Phase A: deionized water 70%, cetrimonium chloride 1%, behentrimonium chloride 2%, cetearyl alcohol 3% polyquaternium-10 0.5%, Phase B: gac oil 0.1%, tamanu oil 0.1%, pomelo peel oil 1.0%, jojoba oil 1%, macadamia oil 0.5%, coconut oil 2%, sunflower seed oil 2%, turmeric root extract 0.1%, dandelion root extract 0.1%, chum ket extract 0.1%, soapnut extract 0.1%, Phase C: vegabead argan 5% (ESP, Naples, Fla.), preservative and fragrance as desired, deionized water q.s. 100%. Instructions: Phase A is heated to 75° C. and sheared to fully disperse components, then heat is removed. When cooled to 45° C., Phase B is added and sheared to disperse components. Finally Phase C is added and mixed thoroughly.

### Example 3: Model Leave-In Curl-Defining Styling Product Formulation

Phase A: deionized water 60%, glycerin 5%, viscarin PC-389 carrageenan 0.1%, xanthan gum 0.1%, Phase B: cetrimonium chloride 1%, behentrimonium chloride 1.8%, cetearyl alcohol 3% polyquaternium-10 0.5%, gac oil 0.1%, tamanu oil 0.2%, pomelo peel oil 0.5%, jojoba oil 1%, macadamia oil 0.5%, coconut oil 2%, sunflower seed oil 2%, turmeric root extract 0.1%, dandelion root extract 0.1%, chum ket extract 0.1%, soapnut extract 0.1%, Phase C: vegabead argan 7% (ESP, Naples, Fla.), vegabead silicone 7% (ESP, Naples Fla.) preservative and fragrance as desired, deionized water q.s. 100%. Instructions: Phase A is heated to 75° C. and sheared to fully disperse components, then heating is stopped. While still hot, Phase B is added and sheared to disperse components. Finally, when cooled to 45° C. Phase C is added and mixed thoroughly.

### Example 4: Model Moisturizing Lotion Product Formulation

Phase A: deionized water 70%, viscarin PC-389 carrageenan 0.1%, xanthan gum 0.1%, avicel PC 815 cellulose gum and sodium alginate 0.3%, turmeric root extract 0.1%, dandelion root extract 0.1%, chum ket extract 0.5%, soapnut extract 0.5%, lauryl glucoside 0.5%, sorbitan monooleate 0.5%, Phase B gac oil 0.1%, tamanu oil 0.1%, pomelo peel oil 2%, jojoba oil 5%, macadamia oil 0.5%, coconut oil 10%, sunflower seed oil 2%, cetyl alcohol 2%, Phase C: deionized water q.s. 100%. Instructions: Phase A ingredients are combined and propeller mixed for 30 minutes to disperse and hydrate gums. Phase B ingredients are combined and heated to 50° C. to melt and incorporate cetyl alcohol component into oil phase. Phase B is added to stirring phase A and sheared with a propeller mixer or homogenizer to form a smooth white emulsion. Preservative and/or fragrance may be included as desired.

The foregoing Examples are intended to describe specific aspects of the composition and methods of making and use thereof, and are not meant to be limiting in any way.

What is claimed:

1. A composition suitable for treating hair, skin or nails, comprising:
a chum ket extract;
a soapnut extract; and
at least one oil or extract selected from the group consisting of gac (Momordica cochinchinensis) oil, tamanu nut oil, lemon peel oil, pomelo peel oil (Citrus maxima peel oil), jojoba oil, macadamia oil, coconut oil, sunflower seed oil, argan oil, turmeric root extract, and dandelion root extract.

2. The composition of claim 1, which comprises at least two of the oil or extract.

3. The composition of claim 1, which comprises at least three of the oil or extract.

4. The composition of any of claim 1, which comprises at least four of the oil or extract.

5. The composition of claim 1, further comprising at least one alginate/chitosan microcapsule.

6. The composition of claim 1, comprising at least 0.1% by weight of the oil or extract.

7. The composition of claim 1, comprising at least 0.5% by weight of the oil or extract.

8. The composition of claim 1, comprising at least 1% by weight of the oil or extract.

9. The composition of claim 1, comprising at least 0.01% by weight, in total, of the chum ket extract and the soapnut extract.

10. The composition of claim 1, which is a shampoo or bodywash.

11. The composition of claim 1, which is a conditioner.

12. The composition of claim 1, which is a leave-in hair product.

13. The composition of claim 1, wherein the composition further comprises a styling product.

14. The composition of claim 1, wherein the composition further comprises at least one of a body oil, cream, lotion, or butter.

15. A method of preparing the composition of claim 1, comprising combining the chum ket extract, soapnut extract, and the at least one oil or extract.

16. A method of treating hair comprising applying an effective amount of the composition of claim 1 to the hair.

17. A method of strengthening hair comprising applying an effective amount of the composition of claim 1 to the hair.

18. A method of reducing and/or preventing hair breakage comprising applying an effective amount of the composition of claim 1 to the hair.

19. A method of treating skin, comprising applying an effective amount of the composition of claim 1 to the skin.

20. A method of treating nails, comprising applying an effective amount of the composition of claim 1 to the nails.
